SEQUENCE LISTING (5 sequences)

SEQ ID NO: 1 - protein, 123 aa, Artificial Sequence
  anti-Her2 VHH
QVQLVQSGGGLVQAGGSLRLSCAASGRTFSSYAMAWFRQAPGKEREFVAAISWSGANIYVADSVKGRFTISRDNAKDTVYLQMNSLKPEDTAVYYCAVKLGFAPVEERQYDYWGQGTQVTVSS

SEQ ID NO: 2 - protein, 126 aa, Artificial Sequence
  anti-Her2-VHH
QVQLQESGGGSVQAGGSLRLSCRASGFTFSSYAMSWVRQPPGKELEWVSAIWGGGDSQHYADSVKGRFTISRDNAKNTLYLQLNSLKTEDTGMYYCVKDRGPFFSGSKYVLGAADRGQGTQVTVSS

SEQ ID NO: 3 - protein, 339 aa, Artificial Sequence
  Anti-CD3-VL-CL-anti-Her2 VHH1
EIVLTQSPATLSLSPGERATLSCSASSGVNYMHWYQQKPGQAPRRWIYDTSKLASGVPARFSGSGSGTSYSLTISSLEPEDFAVYYCHQRGGSYTFGGGTKLEIKRKLTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSQVQLVQSGGGLVQAGGSLRLSCAASGRTFSSYAMAWFRQAPGKEREFVAAISWSGANIYVADSVKGRFTISRDNAKDTVYLQMNSLKPEDTAVYYCAVKLGFAPVEERQYDYWGQGTQVTVSS

SEQ ID NO: 4 - protein, 360 aa, Artificial Sequence
  Anti-CD3-VH-CH1--(GGGGS)3-anti-Her2 VHH1
QVQLVQSGAEVKKPGASVKVSCKTSGYTFTSNWMHWVRQAPGQGLEWMGEIDPSDSYTNYNQKFKGRVTITVDKSASTAYMELSSLRSEDTAVYYCARGSNPYYYAMDYWGQGTLVTVSSKLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGSGGGGSGGGGSGGGGSQVQLVQSGGGLVQAGGSLRLSCAASGRTFSSYAMAWFRQAPGKEREFVAAISWSGANIYVADSVKGRFTISRDNAKDTVYLQMNSLKPEDTAVYYCAVKLGFAPVEERQYDYWGQGTQVTVSS

SEQ ID NO: 5 - protein, 372 aa, Artificial Sequence
  Anti-CD3-VH-CH1-(GGGGS)3-anti-Her2-VHH2
QVQLVQSGAEVKKPGASVKVSCKTSGYTFTSNWMHWVRQAPGQGLEWMGEIDPSDSYTNYNQKFKGRVTITVDKSASTAYMELSSLRSEDTAVYYCARGSNPYYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCAAGGGGSGGGGSGGGGSQVQLQESGGGSVQAGGSLRLSCRASGFTFSSYAMSWVRQPPGKELEWVSAIWGGGDSQHYADSVKGRFTISRDNAKNTLYLQLNSLKTEDTGMYYCVKDRGPFFSGSKYVLGAADRGQGTQVTVSS

(19) United States
(12) Patent Application Publication
(10) Pub. No.: US 2022/0213216 A1
(43) Pub. Date: Jul. 7, 2022

(54) BISPECIFIC ANTIBODY WITH DOUBLE HER2 SITES FOR TUMOR IMMUNOTHERAPY

(71) Applicant: SOUND BIOPHARMACEUTICALS CO. LTD., Chengdu (CN)

(72) Inventors: Qing Li, Chengdu (CN); Fanxin Ma, Chengdu (CN)

(73) Assignee: SOUND BIOPHARMACEUTICALS CO. LTD., Chengdu (CN)

(21) Appl. No.: 17/607,807

(22) PCT Filed: Apr. 27, 2020
(86) PCT No.: PCT/CN2020/087219

§ 371 Date: Oct. 29, 2021

(30) Foreign Application Priority Data
Apr. 30, 2019 (CN) 201910364113.2

(51) Int. Cl. C07K 16/32; C07K 16/28; A61P 35/00

(52) U.S. Cl. CPC C07K 16/32; C07K 16/2809; A61P 35/00; A61K 39/00; C07K 2317/56; C07K 2317/55; C07K 2317/31; C07K 2317/522

(57) ABSTRACT

Disclosed is a bispecific antibody with dual Her2 binding sites, comprising (a) an anti-CD3 antigen-binding fragment Fab, having a light chain variable region VL, a light chain constant region CL, a heavy chain variable region VH and a heavy chain constant region CH1; (b) an anti-Her2 single domain antigen-binding fragment VHH1, linked to the C-terminus of the CL of the Fab and can bind to a first Her2 epitope; and (c) an anti-Her2 single domain antigen-binding fragment VHH2, linked to the C-terminus of CH1 of the Fab and can bind to a second Her2 epitope; the first Her2 epitope and the second Her2 epitope are non-overlapping epitopes of Her2. The bispecific antibody has a killing effect on Her2 tumors with an IHC score of +1, and is effective on trastuzumab-resistant tumors.

### TECHNICAL FILED

The present disclosure relates to a bispecific antibody for tumor immunotherapy, and in particular, to a bispecific antibody with dual Her2 binding sites. The present disclosure also relates to a pharmaceutical composition comprising the bispecific antibody, a polynucleotide encoding the antibody, an expression vector comprising the polynucleotide, and a host cell comprising the expression vector.

### BACKGROUND

Human epidermal growth factor receptor 2 (Her2, also known as Her2/neu or ErbB2) is a member of the Her family of transmembrane receptor tyrosine kinases. Her2 contains a cytoplasmic tyrosine kinase domain, a single transmembrane domain, and an extracellular domain of approximately 630 amino acids. The extracellular domain contains four different domains (domains I-IV). The Her2 proto-oncogene is overexpressed in 25%-30% of human primary breast tumors and various other human cancers (for example, lung, gastric, oral, and colorectal cancers) and is of functional importance.

The important role of Her2 in the development of breast cancer promotes the development of treatments against Her2. The development of anti-Her2 therapies, trastuzumab, lapatinib, pertuzumab and T-DM1 has brought clinical benefits to Her2-positive patients. Trastuzumab remains the main treatment for Her2-positive breast cancers. However, current therapies still suffer from low response rates and drug resistances. For example, due to primary and acquired drug resistances, only 15%-30% of Her2-positive patients respond to trastuzumab treatment. Trastuzumab has minimal effect on cancer cells with low or moderate Her2 expression in vivo and in vitro. Poor internalization will also lead to resistance in T-DM1 treatment of metastatic breast cancers.

In order to improve the efficacy of antibodies against Her-2, an increasing number of new antibodies targeting Her2 have now been reported, including combination therapies and bispecific antibodies. For example, the combination of trastuzumab, pertuzumab and docetaxel has been approved for the first-line treatment of patients with Her2-positive metastatic breast cancer. Bispecific antibodies targeting Her2 and Her3, or targeting two different Her2 epitopes, or bispecific T engagers targeting Her2 and CD3 are also being developed. However, these antibodies and therapies still have no cytotoxicity or only minimum cytotoxicity to cells with low Her2 expression (for example, MCF7 cells with an IHC score of 1+).

### SUMMARY

The present disclosure provides a therapy for patients who are resistant or non-responsive to a Her2 targeted therapy. The present disclosure further provides a more widely applicable therapy, which is effective to treat tumors with any grade of Her2 over-expression (i.e., Her2 tumors with IHC scores of 3+, 2+, or 1+).

In one aspect, the present disclosure provides a bispecific antibody (also referred to herein as Bp-Bs) with dual Her2 binding sites, the bispecific antibody comprising (a) an anti-CD3 antigen-binding fragment Fab, comprising a variable light chain region VL, a light chain constant region CL, a heavy chain variable region VH, and a heavy chain constant region CH1; (b) an anti-Her2 single domain antigen-binding fragment VHH1, which is linked to the C-terminus of the CL of the Fab and is capable of binding to a first Her2 epitope; and (c) an anti-Her2 single domain antigen-binding fragment VHH2, which is linked to the C-terminus of the CH1 of the Fab and is capable of binding to a second Her2 epitope; wherein, the first Her2 epitope and the second Her2 epitope are non-overlapping epitopes of Her2.

In some embodiments, the VHH1 and/or VHH2 is connected to the Fab through a linker (GGGGS)₃. In some embodiments, amino acid sequences of VHH1 and VHH2 are independently selected from a group consisting of a sequence comprising SEQ ID NO. 1, a sequence comprising SEQ ID NO. 2, and a sequence having more than 70%, preferably at least 75%, 80%, 85%, 90%, 91%, 92%, 93%, 94%, 95%, 96%, 97%, 98%, 99% or higher, identity with any of said sequences. In some embodiments, amino acid sequences of the VHH1 and VHH2 are independently selected from a group consisting of SEQ ID NO. 1, SEQ ID NO. 2, and a sequence having more than 70%, preferably at least 75%, 80%, 85%, 90%, 91%, 92%, 93%, 94%, 95%, 96%, 97%, 98%, 99% or higher, identity with any of SEQ ID NO. 1 or 2. In some embodiments, the anti-CD3 antigen-binding fragment Fab is an antigen-binding fragment from the CD3 monoclonal antibody UCHT1. In some embodiments, the bispecific antibody with dual Her2 binding sites has a molecular weight of 60-100 kDa, such as 60, 65, 70, 75, 80, 85, 90, 95, 100 kDa or any value therebetween. In some embodiments, the bispecific antibody with dual Her2 binding sites has a molecular weight of 79 kDa.

Another aspect of the present disclosure provides a bispecific antibody with dual Her2 binding sites, comprising: a first polypeptide chain comprising a light chain constant region CL of an anti-CD3 Fab, a light chain variable region VL of the anti-CD3 Fab and an anti-Her2 single domain antigen-binding fragment VHH1, wherein the VL, CL, and VHH1 are connected sequentially from the N-terminus to the C-terminus, and a second polypeptide chain comprising a heavy chain constant region CH1 of the anti-CD3 Fab, a heavy chain variable region VH of the anti-CD3 Fab and an anti-Her2 single domain antigen-binding fragment VHH2, wherein the VH, CH1, and VHH2 are sequentially connected from the N-terminus to the C-terminus; w % herein the first polypeptide chain is connected to the second polypeptide chain via a disulfide bond.

In some embodiments, the first polypeptide chain has an amino acid sequence comprising a sequence shown in SEQ ID NO. 3 or a sequence having more than 90%, preferably at least 91%, 92%, 93%, 94%, 95%, 96%, 97%, 98%, 99% or higher, identity with the sequence shown in SEQ ID NO. 3. In some embodiments, the second polypeptide has an amino acid sequence comprising a sequence shown in SEQ ID NO. 5 or a sequence having more than 90%, preferably at least 91%, 92%, 93%, 94%, 95%, 96%, 97%, 98%, 99% or higher, identity with the sequence shown in SEQ ID NO. 5. For example, lysine (K) and leucine (L) can be added sequentially after position 120 and before position 121 in SEQ ID NO. 5.

In another aspect, the present disclosure provides a pharmaceutical composition for tumor immunotherapy, the pharmaceutical composition comprising a therapeutically effective amount of the above-mentioned bispecific antibody with dual Her2 binding sites and a pharmaceutically acceptable carrier.

Yet another aspect of the present disclosure provides use of the bispecific antibody with dual Her2 binding sites of the present disclosure in the preparation of a drug for treating tumors.

In some embodiments, the tumor is an IHC 1+, 2+, or 3+ Her2 tumor as determined by immunohistochemistry. In some embodiments, the tumor is selected from a group consisting of esophageal cancer, stomach cancer, colon cancer, rectal cancer, pancreatic cancer, lung cancer, breast cancer, cervical cancer, corpus cancer, ovarian cancer, bladder cancer, head and neck cancer, endometrial cancer, osteosarcoma, prostate cancer, and neuroblastoma. In some embodiments, the tumor is a trastuzumab resistant or non-responsive tumor.

The present disclosure further provides a polynucleotide encoding the first polypeptide chain or the second polypeptide chain, a plasmid containing the polynucleotide of the first polypeptide, and a plasmid containing the polynucleotide of the second polypeptide. The present disclosure also provides an expression vector containing both of the plasmids and a host cell containing the expression vector. The manipulation of polynucleotides involves knowledge and experimental operations in the fields of molecular biology, genetic engineering, protein engineering, etc., which are well known to those skilled in the art.

Another aspect of the present disclosure provides a method for treating a tumor, comprising contacting the bispecific antibody with dual Her2 binding sites of the present disclosure with the cells of the tumor. The present disclosure further provides a method for treating a tumor in a subject, comprising administering to the subject a therapeutically effective amount of the bispecific antibody having dual Her2 binding sites of the present disclosure or the pharmaceutical composition of the present disclosure.

The bispecific antibody with dual Her2 binding sites disclosed herein has one or more of the following advantages.

Compared with bispecific antibodies engineered with intact IgG, bispecific antibodies designed based on Fab structure can reduce the probability of heterologous light chain mismatches during expression due to the existence of two light chains and two heavy chains, simplifying subsequent purification process caused by mismatched products and reducing production costs.

The bispecific antibody designed based on the Fab structure and the VHH structure has a molecular weight of about 79 kDa, which can enhance the permeability of the antibody in the tumor tissue, reduce the spatial limitation of the antibody binding to the target, reduce the possibility of direct excretion by the kidney, and extend the antibody retention time in the body.

Two single-domain antibodies binding to different sites of Her2 were used to build the Bp-Bs disclosed herein, which can improve the ability of the antibody to bind to Her2 positive tumors, play a synergistic effect of targeting Her2, and show an effect on tumors with Her2 weak expression.

The present antibody shows a different mechanism of action from trastuzumab, and can treat trastuzumab-resistant tumors.

The C-terminus of the heavy chain constant region CH1 of the anti-CD3 Fab is connected to another single-domain antibody via a non-polar hydrophobic flexible peptide (GGGGS)₃, which can improve the spatial flexibility of the binding of the two single-domain antibodies at the C-terminus of the Fab to the antigen.

### BRIEF DESCRIPTION OF THE DRAWINGS

FIGS. 1A-C. Schematic diagram of the structures of Bp-Bs and Bi-Bs and their binding modes with Her2. A. Bi-Bs; B. Bp-Bs; C. binding mode. FIG. 1D shows the SDS-PAGE electrophoresis of Bp-Bs and Bi-Bs under reducing and non-reducing conditions.

FIG. 2A. Flow cytometry detecting the binding of Bp-Bs and Bi-Bs to CHO, MCF7, LS174T and SKOV3 cells. FIG. 2B. Confocal laser microscopy detecting the localization of Bp-Bs and Bi-Bs on the surface of CHO and SKBR3 cells. FIG. 2C. Affinity constants of Bp-Bs and Bi-Bs binding to Her2 antigen.

FIG. 3. Bp-Bs and Bi-Bs promote T cell-mediated cytotoxic killing. A. The effect of different concentrations of antibodies on tumor cells, in the presence or absence of T cells; B. Dose-dependent cytotoxicity killing experiment. All data are shown as mean and standard deviation of three replicate samples (***P<0.001 vs. tumor cells plus T cell group, Dunnett's multiple comparisons test).

FIG. 4. Bp-Bs has a weak effect on the downstream signaling pathway of Her2. Different tumor cells were incubated with antibodies for 30 hours, and then the total protein of the cell lysate was extracted for western blotting. A. SKOV3 cells; B. LS174T cells; C. MCF7 cells.

FIG. 5. Pharmacokinetic characteristics of Bp-Bs and Bi-Bs in mice. Top: concentrations of Bp-Bs and Bi-Bs in serum after intravenous bolus. The result is the mean and standard deviation of three replicate samples. Bottom: pharmacokinetic parameters. Cmax: highest plasma concentration; AUC all: area under the drug-time curve; CL: total clearance; Vss: apparent volume of distribution; t1/2: elimination half-life.

FIG. 6. Anti-tumor activity of Bp-Bs in LS174T human colon cancer transplantation model. A. The growth inhibitory effect of different drugs on tumors. B. Changes in body weight of mice in each experimental group after administration. The results are the mean and standard error of 6 mice in each group (***P<0.001, Dunnett's multiple comparisons test, vehicle vs Trastuzumab and vehicle vs Bp-Bs; ***P<0.001, paired t test, Trastuzumab vs Bp-Bs).

FIG. 7. Bp-Bs has stronger tumor inhibitory activity than Bi-Bs. A. The inhibitory effect of different drugs on tumor growth; B. Anatomy of mice subcutaneous tumor at 14 days after the administration; C. The weight change of the mice in each experimental group after the administration. The results are the mean and standard error of 5 mice in each group. (**P<0.01, vehicle vs Bi-Bs; ***P<0.001, vehicle vs Bp-Bs; Dunnett's multiple comparisons test; *P<0.05, paired t test, Bi-Bs vs Bp-Bs).

### DETAILED DESCRIPTION OF THE EMBODIMENTS

### Definition

“Antibody” as used herein refers to any form of antibody that exhibits the desired biological activity, for example, inhibiting the binding of a ligand to its receptor or inhibiting receptor signal transduction induced by the ligand. Therefore. “antibody” has its broadest meaning in the present disclosure, and clearly includes but is not limited to monoclonal antibodies (including full-length monoclonal antibodies), polyclonal antibodies, and multi-specific antibodies (such as bispecific antibodies).

As used herein, the term “composition” refers to a formulation suitable for administration to an intended animal subject for therapeutic or preventive purposes, which contains at least one pharmaceutically active ingredient, such as a compound.

As used herein, the terms “therapeutically effective amount” and “effective amount” indicate the substance and the amount of the substance are effective in preventing, reducing or improving one or more symptoms of a disease or disorder, and/or prolonging the survival of a subject receiving the treatment.

“Treatment” as used herein includes administering a compound of the present application, a pharmaceutically acceptable salt, or composition thereof, to reduce the symptoms or complications of the disease or disorder, or to eliminate the disease or disorder. As used herein, the term “alleviation” is used to describe the process of reducing the severity of the signs or symptoms of a disorder. Symptoms can be alleviated but not eliminated. In one embodiment, administration of the pharmaceutical composition of the present disclosure results in the elimination of the signs or the symptoms.

“Subject” or “individual” or “animal” or “patient” or “mammal” refers to any subject for which diagnosis, prognosis or treatment is desired, especially a mammalian subject. A mammalian subject includes humans, domestic animals, farm animals, zoo animals, sports animals or pets, such as dogs, cats, guinea pigs, rabbits, rats, mice, horses, bovine, cows, and the like.

As demonstrated in the examples, an exemplary bispecific antibody is an antibody that targets two different antigens, one of which is present on tumor cells or microorganisms, and the other is on immune cells. When administered to an individual, the bispecific antibody specifically binds to the tumor cells or the microorganisms, and at the same time specifically binds to the immune cells (such as cytotoxic cells). This dual binding can cause the bound tumor cell or microorganism to be killed by the host's immune system.

The term “single domain antigen-binding fragment” or “single domain antibody fragment” or “VHH” is an antigen-binding fragment capable of binding to an antigen without being equipped with a light chain. VHH was originally isolated from a single domain antibody (sdAb) as a single antigen-binding fragment. The first known single domain antibody was isolated from camel and later from cartilaginous fish. Camels produce functional antibodies without light chains, and their single N-terminal domain (VHH) binds to antigen without domain pairing. Single domain antibodies do not include the CH1 domain, which interacts with the light chain in conventional antibodies.

VHH contains four framework regions (FR1-FR4) that constitute the core structure of an immunoglobulin domain and three complementarity determining regions (CDR1-CDR3) involved in antigen binding. The VHH framework region shows high sequence homology (>80%) with the human VH domain. It was reported that the most characteristic feature of VHH lies in the amino acids at four FR2 positions, i.e., positions 37, 44, 45, and 47 (Kabat numbering), which are conserved in the conventional VH domain and involved in hydrophobic interaction with the VL domain. VHH usually has different amino acids at these and other positions that are highly conserved in conventional VH (such as Leu11Ser, Val37Phe, Val37Tyr, Gly44Glu, Leu45Arg, Leu45Cys, or Trp47Gly).

The extracellular domain of Her2 includes four domains, domain I (ECD1, about 1-195 amino acid residues), domain II (ECD2, about 196-319 amino acid residues), domain III (ECD3, about 320-488 amino acid residues) and domain IV (ECD4, about 489-630 amino acid residues) (residue numbering, no signal peptide). Those skilled in the present invention can select the epitopes of Her2 by methods known in the art, and determine the VHH fragment that binds to the epitope according to known methods.

The terms “Her2 positive tumor”, “Her2 overexpression tumor” or similar refer to tumor diseases characterized by overexpression of Her2 protein or amplification of Her2 gene. The term “overexpression” of Her2 protein refers to the abnormal level of expression of Her2 receptor protein in cells derived from a tumor in a specific tissue or organ of a patient, relative to the expression level in normal cells derived from the tissue or organ. Patients or subjects with cancer characterized by overexpression of the Her2 receptor can be determined by standard assays known in the art. Her2 positive cancers specifically refer to cancers with overexpression of Her2 of degree 1+(Her2 1+), degree 2+(Her2 2+), or degree 3+(Her2 3+) as determined by immunohistochemistry.

In certain embodiments, Her2 positive cancers are cancers with Her2 expression of 2+ or lower, preferably 1+ or lower, as determined by immunohistochemistry. As shown by the examples, patients with cancers characterized by Her2 protein overexpression in the range of 1+, 2+, or 3+, preferably 1+ or 2+, more preferably 1+ or lower will benefit from the treatment method disclosed herein. In this respect, immunohistochemistry refers to the immunohistochemical staining of fixed tumor samples and the analysis of the staining. Her2 expression level 0 (Her2 0) refers to no staining or cell membrane staining in less than 10% of tumor cells, especially less than 20,000 Her2/cells. Her2 1+ refers to weak staining of the cell membrane in more than 10% of tumor cells, where the cell membrane is only partially stained, especially about 100,000 Her2/cells. Her2 2+ means that the entire cell membrane is weakly to moderately stained in more than 10% of tumor cells, especially about 500,000 Her2/cells. Her2 3+ means that the intact cell membrane is strongly stained in more than 10% of tumor cells, especially about 2,000,000 Her2/cells.

Preferably, a histological sample containing cancer cells, especially a formalin-fixed paraffin-embedded cancer tissue sample, is used to determine the expression of Her2. The immunohistochemical assay for determining Her2 overexpression preferably includes (i) contacting a sample containing cancer cells with a first antibody directed against Her2, and then (ii) contacting the sample with a second antibody against the first antibody and conjugated with a visualization agent, such as an enzyme that catalyzes a reaction with a visible end product, such as horseradish peroxidase. Suitable Her2 immunohistochemistry kits are HercepTest (Dako Denmark A/S) and Pathway Her2 (Ventana Medical Systems, Inc.).

Her2 positive tumor diseases also include cancers that are positive for Her2 gene amplification as determined by fluorescence in situ hybridization (FISH) or chromogenic in situ hybridization (CISH). According to the FISH assay, if the copy number of the Her2 gene in the tumor cell is at least twice the copy number of chromosome 17 or if the tumor cell contains at least 4 copies of the Her2 gene, the cancer is positive for the Her2 gene duplication. According to the CISH assay, if at least 5 copies of the Her2 gene per nucleus are present in at least 50% of the tumor cells, the cancer is positive for the Her2 gene duplication.

Cells expressing Her2, for example breast cancer cell lines, can be used to evaluate the antibodies disclosed herein. The following table describes the expression levels of Her2 in several representative cancer cell lines.

| Cell lines | Description | IHC Scores | Her2 receptor/cell |
|---|---|---|---|
| NCI-N87 | human gastric cancer | 3+ | Not Evaluated |
| A549 | human alveolar carcinoma | 0/1+ | Not Evaluated |
| | (non-small cell lung cancer) | | |
| BxPC-3 | human pancreatic cancer | 1+ | Not Evaluated |
| MIAPaCa-2 | human pancreatic ductal | 2+ | Not Evaluated |
| | adenocarcinoma | | |
| FaDu | human pharyngeal squamous | 2+ | Not Evaluated |
| | cell carcinoma | | |
| HCT-116 | human colorectal epithelial | 1+ | Not Evaluated |
| | carcinoma | | |
| WI-38 | normal fatal lung | 0+ | 1.0 × 10E4 |
| MDA-MB- | human triple negative breast | 0/1+ | 1.7 × 10E4 − 2.3 × 10E4 |
| 231 | epithelial adenocarcinoma | | |
| MCF-7 | human estrogen receptor | 1+ | 4 × 10E4 − 7 × 10E4 |
| | positive breast epithelial | | |
| | adenocarcinoma | | |
| JIMT-1 | Trastuzumab-resistant breast | 2+ | 2 × 10E5 − 8 × 10E5 |
| | epithelial cancer, amplified | | |
| | HER2 oncogene, insensitive to | | |
| | HER2 inhibitory drug (i.e., | | |
| | Herceptin ™) | | |
| ZR-75-1 | estrogen receptor positive | 2+ | 3 × 10E5 |
| | breast ductal carcinoma | | |
| SKOV-3 | human ovarian epithelial | 2/3+ | 5 × 10E5 − 1 × 10E6 |
| | adenocarcinoma, HER2 gene | | |
| | amplification | | |
| SK-BR-3 | human breast epithelial | 3+ | >1 × 10E6 |
| | adenocarcinoma | | |
| BT-474 | human breast epithelial ductal | 3+ | >1 × 10E6 |
| | carcinoma | | |

The term “trastuzumab-resistant tumor” is defined as a decrease in the sensitivity of tumor cells to trastuzumab. Patients with such tumors are identified as “trastuzumab-resistant tumor” patients. Since the resistance can be primary or acquired, any observed decrease in sensitivity is compared with full sensitivity of “normal” tumor cells (responds to the anti-tumor drug applied in relative to their initial sensitivity at the beginning of treatment). In the latter case, the resistance is manifested as a decrease in the amount of tumor regression at the same dose or an increase in the dose necessary for the same amount of tumor regression.

As used herein, “inhibition” or “treatment” includes delaying the development of symptoms associated with a disease and/or reducing the severity of these symptoms that the disease will or expected to develop. The term also includes alleviating existing symptoms, preventing additional symptoms, and alleviating or preventing the underlying causes of these symptoms. Therefore, the term means that a beneficial result has been conferred on a vertebrate subject suffering from a disease.

The term “therapeutically effective amount” or “effective amount” as used herein refers to when the bispecific antibody having dual Her2 binding sites or a fragment thereof disclosed herein is administered alone or in combination with another therapeutic agent to a cell, tissue or subject, it effectively prevents or slows the amount of the disease or condition to be treated. A therapeutically effective dose further refers to the amount of the antibody sufficient to cause alleviation of symptoms, such as treating, curing, preventing or alleviating related medical conditions, or improving the treatment rate, cure rate, prevention rate, or alleviation rate of the symptoms. When administered to an individual alone, the therapeutically effective amount refers to the mount of the alone ingredient. When a combination is administered, the therapeutically effective amount refers to the combined amount of active ingredients that produce a therapeutic effect, regardless of whether it is administered in combination, sequentially or simultaneously. A therapeutically effective amount will reduce symptoms usually by at least 10%; usually at least 20%; preferably at least about 30%; more preferably at least 40% and most preferably at least 50%.

### Pharmaceutical Preparations and Pharmaceutical Compositions

The present disclosure includes a pharmaceutical preparation of the bispecific antibody or antibody fragment with double Her2 binding sites disclosed herein. To prepare a pharmaceutical composition or a sterile composition, the antibody or fragment thereof is mixed with a pharmaceutically acceptable carrier or excipient. The therapeutic and diagnostic preparation of drugs can be prepared in the form of, for example, lyophilized powder, slurry, aqueous solution or suspension by mixing with physiologically acceptable carriers, excipients or stabilizers.

The toxicity and therapeutic efficacy of antibody compositions administered alone or in combination with immunosuppressive agents can be measured in cell cultures or experimental animals by standard pharmaceutical methods, such as methods for determining LD₅₀ (dose causing 50% of the population lethal) or ED₅₀ (dose effective to treat 50% of the population). The dose ratio between toxic and therapeutic effects is the therapeutic index, which can be expressed as the ratio of LD₅₀ to ED₅₀. The data obtained from these cell culture assays and animal studies can be used to formulate a range of dosages for use in humans. The dosage of the antibody is preferably within a range of circulating concentrations that include the ED₅₀ with little or no toxicity. The dosage can be varied within this range according to the dosage form used and the route of administration used.

Suitable routes of administration include parenteral administration (for example, intramuscular, intravenous or subcutaneous administration) and oral administration. The antibody used in the pharmaceutical composition or for practicing the method of the present invention can be administered in a variety of conventional ways, such as oral ingestion, inhalation, topical application or transdermal, subcutaneous, intraperitoneal, parenteral, intraarterial or intravenous injection. In one embodiment, the antibody of the invention is administered intravenously. In another embodiment, the antibody of the invention is administered subcutaneously. Alternatively, one can administer the antibody in a local rather than systemic manner (usually a long-acting or sustained-release formulation), for example via injection of the antibody directly to the site of action. In addition, one can administer the antibody in a targeted drug delivery system.

The appropriate dose is determined by the clinician, for example, using parameters or factors known or suspected to affect the treatment or expected to affect the treatment in the art. Generally, the starting dose is slightly lower than the optimal dose, and thereafter a small increase until the desired or optimal effect relative to any adverse side effects is achieved. Important diagnostic measures include measuring, for example, inflammatory symptoms or the level of inflammatory cytokines produced.

The antibodies and antibody fragments can be administered by continuous infusion or by dosing at regular intervals, for example, one day, one week, or 1-7 times a week. The dose can be provided intravenously, subcutaneously, intraperitoneally, transdermally, topically, orally, nasally, transrectally, intramuscularly, intracerebrally, intraspinally, or by inhalation. A preferred dosage regimen is a regimen that includes the maximum dosage or dosing frequency that avoids significant undesirable side effects. The total weekly dose is usually at least 0.05 μg/kg body weight, more usually at least 0.2 μg/kg, most usually at least 0.5 μg/kg, typically at least 1 μg/kg, more typically at least 10 μg/kg, most typically at least 109 μg/kg, preferably at least 0.2 mg/kg, more preferably at least 1.0 mg/kg, most preferably at least 2.0 mg/kg, ideally at least 10 mg/kg, more ideally at least 25 mg/kg, and most ideally at least 50 mg/kg. Based on mol/kg calculation, the required dose of small molecule therapeutics such as peptide mimetics, natural products or organic chemical agents is approximately the same as the dose of antibodies or polypeptides.

The pharmaceutical composition disclosed herein may also contain other agents, including but not limited to cytotoxic agents, cell growth inhibitors, anti-angiogenic drugs or antimetabolites, targeted tumor drugs, immunostimulants or immunomodulators, or antibodies conjugated to cytotoxic agents, cell growth inhibitors or other toxic drugs. The pharmaceutical composition can also be administered with other treatment modalities such as surgery, chemotherapy, and radiation. Typical veterinarians, experiments or research subjects include monkeys, dogs, cats, rats, mice, rabbits, guinea pigs, horses, and humans.

### Tumors

The antibodies disclosed herein can be used to treat tumors, i.e., inhibit the growth or survival of tumor cells. Preferred tumors whose growth can be inhibited by the antibody disclosed herein include tumors that generally respond to immunotherapy. Non-limiting examples of preferred cancers for treatment include Her2 overexpression cancers. The Her2 overexpression cancers may include Her2 high overexpression cancers, Her2 medium overexpression cancers, or Her2 low overexpression cancers. Examples of Her2 overexpression cancers include, but are not limited to, esophageal cancer, stomach cancer, colon cancer, rectal cancer, pancreatic cancer, lung cancer, breast cancer, cervical cancer, corpus carcinoma, ovarian cancer, bladder cancer, head and neck cancer, endometrial cancer, osteosarcoma, prostate cancer, and neuroblastoma. As mentioned above, Her2 overexpression cancers can be classified into Her2 1+, Her2 2+, and Her2 3+ overexpression cancers according to IHC. The antibody disclosed herein is suitable for treatment of Her2 1+, Her2 2+, and Her2 3+ overexpression cancers. Experiments have confirmed that the antibody disclosed herein still has a significant killing effect on Her2 1+ tumors.

The antibody disclosed herein can be used alone or in combination with the following substances: anti-tumor drugs or immunogenic agents, such as attenuated cancer cells; tumor antigens including recombinant proteins, peptides and carbohydrate molecules; antigen-presenting cells, such as tumor-derived dendritic cells stimulated by the antigen or nucleic acid, immunostimulatory cytokines (such as IL-2, IFNa2, GM-CSF) and cells transfected with genes encoding immunostimulatory cytokines (such as but not limited to GM-CSF); standard cancer treatment (such as chemotherapy, radiotherapy or surgery); or other antibodies, including but not limited to antibodies against the following antigens: VEGF, EGFR, VEGF receptors, other growth factor receptors, CD20, CD40, CTLA-4, OX-40, 4-IBB and ICOS, such as trastuzumab or patolizumab.

### Combinational Therapy

As described above, the bispecific antibody with dual Her2 binding sites disclosed herein can be co-administered with one or more other therapeutic agents, such as cytotoxic agents, radiotoxic agents or immunosuppressive agents. The antibody can be conjugated to the agent as an immune complex, or can be administered separately from the therapeutic agent. In the latter case (separate administration), the antibody can be administered before, after or concurrently with the therapeutic agent, or it can be co-administered with other known therapies.

The antibodies can also be used in in vivo diagnostic assays. The antibody is usually labeled with a radionuclide, such as ¹¹¹In, ⁹⁹Tc, ⁴C, ³¹I, ¹²⁵I, ³H, ³P, ³⁵S or ¹⁸F, so that immunoimaging or positron imaging can be used to locate the antigen or antigen-expressing cells.

The present invention will be more fully understood by referring to the following examples. However, these examples should not be construed as limiting the scope of the present invention. All documents and patent citations mentioned herein are expressly incorporated herein by reference.

### EXAMPLES

### Example 1. Design and Purification of Bispecific Antibody Bp-Bs and its Control Bi-Bs

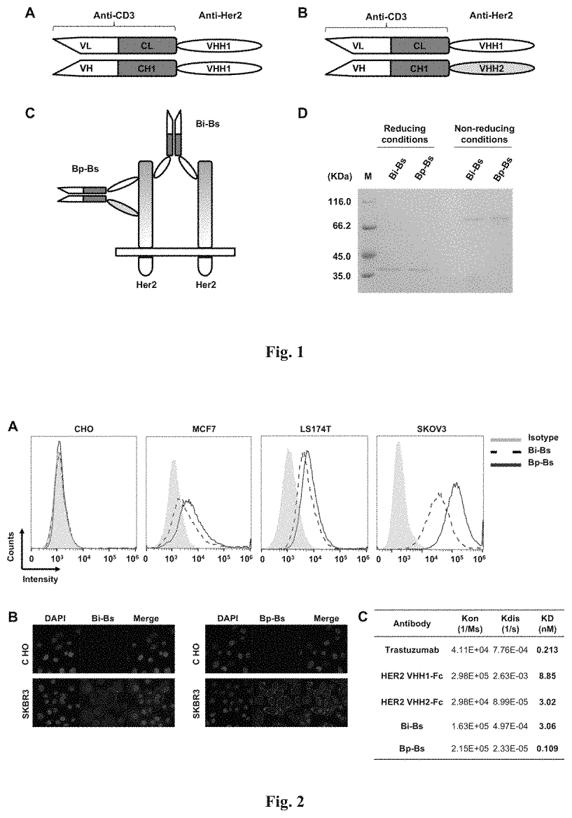

The structures of the bivalent anti-Her2 bispecific antibody (Bi-Bs) and the bispecific antibody Bp-Bs that binds to Her2 dual sites are shown in FIGS. 1A and 1B, respectively. DNA shuffling and ligation techniques were used to clone the respective genes. Bi-Bs: single-chain domain anti-Her2 VHH1 (SEQ ID NO. 1, GenBank: JX047590.1; Even-Desrumeaux, K., P. Fourquet, V Secq, D. Baty and P. Chames (2012). “Single-domain antibodies: a versatile and rich source of binders for breast cancer diagnostic approaches.” Mol Biosyst 8(9): 2385-2394.) was linked to the C-termini of the VH-CH1 and the VL-CL of the anti-CD3 UCHT1 clone (with linker: (GGGGS)₃): Bp-Bs: the anti-Her2 VHH1 at the VH-CH1 of Bi-Bs was replaced with another anti-Her2 VHH2 (SEQ ID NO.2; Wu, X., S. Chen, L. Lin, J. Liu, Y. Wang, Y. Li, et al. (2018). “A Single Domain-Based Anti-Her2 Antibody Has Potent Antitumor Activities.” Transl Oncol 11(2): 366-373.).

The heavy chain and light chain genes were cloned into pET26b vector (heavy chain HC) and pET21a vector (light chain LC). The Bp-Bs antibody was formed by heterodimerization of VH-CH1-VHH2 (SEQ ID NO.5) and VL-CL-VHH1 (SEQ ID NO.3). The Bi-Bs antibody was formed by the heterodimerization of VH-CH1-VHH1 (SEQ ID NO. 3) and VL-CL-VHH1 (SEQ ID NO. 4). The recombinant plasmids obtained by molecular cloning technology were co-transformed into BL21 E. coli competent cells at a ratio of 1:1, and grown on agarose plates with dual resistance to kanamycin and ampicillin to obtain a monoclonal double transformant colony. A single colony was picked and inoculated in LB medium and then expanded to M9 medium. IPTG was added to induce E. coli to express Bi-Bs and Bp-Bs proteins. The supernatant of the medium was collected and purified by Ni Sepharose affinity purification to produce Bi-Bs and Bp-Bs proteins. The purified antibodies Bi-Bs and Bp-Bs were then subjected to SDS-PAGE electrophoresis under reducing and non-reducing conditions, and stained by Coomassie brilliant blue. As shown in FIG. 1D, the relative mobility of the purified proteins on SDS-PAGE is consistent with the expected molecular weight of the single-chain Bi-Bs or Bp-Bs antibody of 39 kDa under reducing condition, and of 79 kDa under non-reducing condition.

### Example 2. Bp-Bs Antibody Binding Characteristics

Methods

Cell lines: CHO, MCF7, LS174T, SKOV3, SKBR3 cells were all from the cell bank of the Type Culture Collection Committee of the Chinese Academy of Sciences; the cell culture medium, fetal bovine serum, pancreatin, penicillin-streptomycin antibiotic mixture and other additives were purchased From Gibco; all consumables for cell culture were purchased from Corning Costar. All cell lines were cultured in DMEM (for MCF7, SKBR-3 and SKOV3) or RPMI-1640 (Thermo, China) (for LS174T and CHO) containing 10% HI fetal bovine serum (Thermo, USA) and 1% penicillin/streptomycin (Hyclone) at 37° C., 5% CO₂.

Affinity assay: The OctetQKe instrument (Pall Life Sciences) was used to determine the affinity of the anti-Her2 antibody to the extracellular region of the Her2 protein. Briefly, human Her2 with an Fc tag in PBST (AcroBiosystem, catalog number HE2-H5253) was loaded onto the surface of ProteinA Capture Biosensors (ProA). Immobilization level was set at 0.8 nM to 1.2 nM. Then a 60-second biosensor baseline step was performed, and the antigen/antibody association was analyzed for 180 seconds on the biosensor to test the antibody/antigen. The test molecules were then applied at a two-fold concentration gradient. The data analysis software version 8.2 (PALL/ForteBio) was used to evaluate the Octet data, and the full-fit 1:1 modal was used to determine the Kd value.

Flow cytometry analysis: Flow cytometry was used to assess the binding of bispecific antibodies on Her2 positive or negative cells. Different cell lines were cultured and resuspended after trypsinization. The cells were then washed and resuspended in 0.1% BSA in PBS. In the absence or presence of antibodies, a total of 100 μL of 5×10⁵ cells per sample were incubated for 1 hour on ice. After washing twice with ice-cold PBS, the cells were incubated with goat anti-human IgG (H+L)-AF488 (Invitrogen, catalog number A11013) for 1 hour on ice. Cytomics FC500 flow cytometer (Beckman Coulter) was used to analyze cell-related fluorescence, and FlowJo (http://www.flowjo.com) was used for graphing.

Immunofluorescence test: In order to further analyze the binding of antibody and Her2 on cell surface, immunofluorescence assay was performed as described previously (Xing, J., L. Lin, J. Li, J. Liu, C. Zhou, H. Pan, et al. (2017). “BiHC, a T-Cell-Engaging Bispecific Recombinant Antibody, Has Potent Cytotoxic Activity Against Her2 Tumor Cells.” Transl Oncol 10(5): 780-785). Briefly. CHO and SKBR3 cells were cultured overnight on a glass bottom culture dish (Cellvis). After washing three times with PBS, the cells were fixed with 4% paraformaldehyde. After blocking with PBS plus 1% BSA for 1 hour at room temperature, the cells were incubated with the antibody for 1 hour at room temperature. After washing three times with PBS, the sample was incubated with goat anti-human IgG (H+L)-AF488 at 4° C. for 1 hour. After washing with PBS, the samples were observed using a confocal laser scanning microscope (Zeiss EC Plan-Neofluar 40×/1.30 Oil DIC M27 objective lens) and analyzed by ZEN software.

Results

In order to detect the binding ability of the antibodies to Her2 antigen, biofilm interference technology (BLI) was used to analyze the interaction between the antibodies and Her2 antigen. As shown in FIG. 2C, trastuzumab (control), anti-Her2-VHH1-Fc, or anti-Her2-VHH2-Fc have an affinity of 0.213 nM, 8.85 nM, and 3.02 nM, respectively. The affinity data (KD) (FIG. 2C) shows that the affinities of the single-site bivalent bispecific antibody Bi-Bs (3.06 nM) modified based on anti-Her2 VHH1 and of the single-site bivalent antibody anti-Her2 VHH1-Fc (8.85 nM) are similar, indicating that the modification of the bispecific antibody does not affect the ability of the antibody to bind to Her2. However, the affinity of the bispecific antibody Bp-Bs (0.109 nM) with dual Her2 binding sites is 30 stronger than that of the single-site bivalent antibody anti-Her2 VHH1-Fc, anti-Her2 VHH2-Fc (3.02 nM) or Bi-Bs, while is equivalent to Trastuzumab, indicating that the antibody Bp-Bs with dual Her2 binding sites has a higher affinity for Her2 antigen.

In order to detect the ability of the antibodies to bind to cell surface antigens, flow cytometry (FACS) was used to analyze Bp-Bs and Bi-Bs. Experiments were conducted with the Her2negative cell line CHO, Her2 high expression cell line SKOV3, Her2 medium expression cell line LS174T and Her2 weak expression cell line MCF7. The results showed (FIG. 2A): Neither Bp-Bs nor Bi-Bs bound to Her2 and CD3 negative cell CHO; in Her2 positive cell lines, the displacement of Bp-Bs and Bi-Bs positive fluorescence signals was positively correlated with Her2 expression, suggesting that Bp-Bs and Bi-Bs had different degrees of binding on SKOV3, LS174T and MCF7 cells; compared with Bi-Bs, Bp-Bs showed stronger binding ability in Her2 positive cells.

Confocal microscopy was then used to analyze the binding of Bp-Bs and Bi-Bs to the Her2 protein on the surface of Her2 positive SKBR3 cells, and Her2 negative CHO cells were used as the control group. After SKBR3 and CHO cells were incubated with Bp-Bs and Bi-Bs, respectively, they showed obvious fluorescent localization on the surface of SKBR3 cell membrane, but no fluorescent localization appeared on the surface of CHO cell membrane, suggesting that Bp-Bs or Bi-Bs can bind to Her2 proteins on the surface of SKBR3 cells, and Bp-Bs has stronger cell binding ability than Bi-Bs (FIG. 2B). The results of flow cytometry and laser confocal together show that Bp-Bs can specifically bind to Her2 positive tumor cells, with a higher binding ability.

### Example 3. Bp-Bs Antibody Induced T Cell-Mediated Cytotoxicity

Methods

In order to determine the cytotoxicity of bispecific antibodies in vitro, Ficoll-Plaque Plus (GE health) gradient centrifugation was used to prepare human peripheral blood mononuclear cells (PBMC) from freshly donated blood. Human peripheral blood was collected from healthy volunteers with written consent. The EasySep™ human CD3 positive selection kit (Stemcell Technologies, Inc., Vancouver, BC, Canada) was used to isolate human CD3+ T cells from PBMC according to the manufacturer's instructions. The cytotoxicity assay was performed as previously described (Li, L., P. He, C. Zhou. L. Jing, B. Dong, S. Chen, et al. (2015). “A novel bispecific antibody, S-Fab, induces potent cancer cell killing.” J Immunother 38(9): 350-356). Briefly, SKOV3, MCF7, LS174T or CHO cancer cells were trypsinized and seeded in a 96-well tissue culture plate at a density of 5,000 cells/well as target cells, and incubated overnight at 37° C., 5% CO₂. Then, 50,000 human CD3+ T cells without pre-stimulation were added as effector cells. Different concentrations of the anti-Her2 antibody were added to different wells. After 72 hours of incubation, cell counting kit-8 reagent (Dojindo, CK04) was used to quantify cell viability according to the manufacturer's protocol. The survival rate (%) of target cells was calculated using the following formula: [(live target cells (sample)−medium)/(live target cells (control)−medium)]×100%.

Results

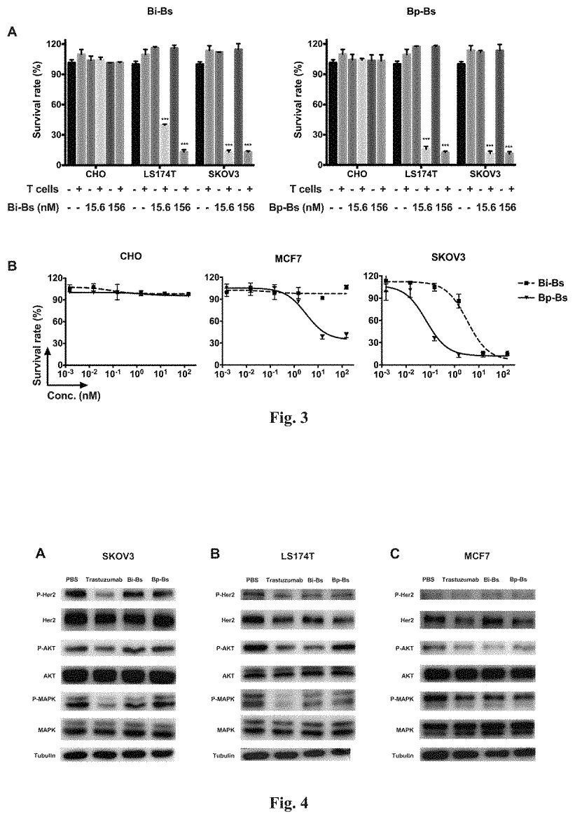

In order to determine whether Bp-Bs and Bi-Bs can recruit T cells to kill Her2 positive tumor cells, cytotoxic killing experiment was performed. The results showed (FIG. 3A) that neither Bp-Bs nor Bi-Bs can recruit T cells to kill Her2 negative cells CHO. In the groups without T cells, high and low concentrations of Bp-Bs and Bi-Bs could not inhibit the growth of Her2 positive cells SKOV3 and LS174T. In the groups with T cells, 15.6 nM and 156 nM of Bp-Bs or Bi-Bs showed significant tumor killing effect on Her2 positive cells. In addition, under the same conditions, low concentration of Bp-Bs recruited T cells had a slightly stronger killing effect on Her2 medium-expressing cells LS174T than Bi-Bs.

In order to further explore the dose-effect relationship of Bp-Bs and Bi-Bs on tumor cell toxicity, the killing effect of gradient concentrations of Bp-Bs and Bi-Bs on tumor cells was evaluated. According to the results of the cytotoxic killing experiment (FIG. 3A), antibody concentration ranges were determined as 1.56×10² nM to 1.56×10⁻³ nM. SKOV3 and MCF7 cells were selected as the target cells of the dose-dependent cytotoxicity killing experiment, and CHO cells were used as the control. The results showed (FIG. 3B) that within a certain concentration range, the cytotoxic effect of Bp-Bs or Bi-Bs was positively correlated with the expression level of Her2 on the surface of tumor cells; and the killing effect of Bp-Bs or Bi-Bs on SKOV3 cells was positively correlated with doses. However, for MCF7 cells, the highest concentration of Bi-Bs still had no obvious killing effect. When the dose of Bp-Bs was greater than 1.56 nM, the killing effect starts to appear, and it was positively correlated with the dose. These results indicate that, compared with Bi-Bs, Bp-Bs recruited T cells to specifically kill Her2 positive tumor cells, especially tumor cells with weak Her2 expression.

### Example 4. Bp-Bs Recruited T Cell for the Treatment of Her2 Positive Tumors Through the Her2 Dual Site Design

Methods: SKOV3, LS174T or MCF7 cells were inoculated (300,000 cells/well) in a 6-well plate, and incubated overnight at 37° C. The cells were then treated with or without 100 nM anti-Her2 antibody at 37° C. for 30 hours. After incubation, the cells were washed twice with cold PBS and lysed using RIPA lysis buffer (Beyotime, catalog number P0013B) according to the manufacturer's instructions. The protein concentration was determined by the BCA method (Thermo Fisher Scientific), and each 20 μg protein sample was analyzed by 8% SDS-PAGE, and subjected to western blotting using antibodies against ErbB2, phospho-ErbB2-Tyr1221/1222, AKT, phospho-AKT-Ser473, p44/42 MAPK, phospho-p44/42 MAPK-Thr202/Tyr204 and Tubulin (Cell Signaling Technology, catalog numbers 4290, 2243, 4691, 4060, 4695, 9101 and 2144).

Results: One of the mechanisms of the clinically used Her2 monoclonal antibody trastuzumab to inhibit tumor growth is to inhibit the expression of Her2 protein and down-regulate the downstream PI3K signaling pathway of Her2. The results showed (FIG. 4) that trastuzumab can inhibit Her2 protein expression and Her2 protein phosphorylation in LS174T and MCF7 cells, and down-regulate the phosphorylation levels of MAPK and AKT proteins in the downstream signaling pathways of Her2 (FIGS. 3B and C). Compared with trastuzumab, Bp-Bs or Bi-Bs can only slightly down-regulate the phosphorylation of Her2 and MAPK proteins in SKOV3, LS174T and MCF7 cells, suggesting that Bp-Bs and Bi-Bs have weaker effects on the downstream signaling pathways of Her2. The anti-tumor mechanism of Bp-Bs and Bi-Bs mainly relies on its anti-CD3 Fab fragment to recruit T cells to kill tumors. Therefore, Bp-Bs can be used for the treatment of trastuzumab-resistant tumors.

### Example 5. In Vivo Pharmacokinetics of Bp-Bs

Methods: pharmacokinetic (PK) study: A single-dose PK study of Bi-Bs and Bp-Bs was conducted in female CB-17 SCID mice. The animals were randomly divided into different treatment groups (n=9 in each group, 3 animals at each time point), and 1 mg/kg Bi-Bs or Bp-Bs was injected intravenously. Serum samples were collected 0.25, 0.5, 1, 2, 4, 8, 12, 24, and 48 hours after injection for bioanalytical measurement. The antibody concentrations in serum were determined by ELISA as previously reported (Pan, H., J. Liu, W. Deng, J. Xing, Q. Li and Z. Wang (2018). “Site-specific PEGylation of an anti-CEA/CD3 bispecific antibody improves its antitumor efficacy.” Int J Nanomedicine 13: 3189-320.). Kinetica (v.5.1 SP1, Thermo Fisher Scientific) was used to analyze the blood drug concentration data obtained at each time point in a non-compartmental model.

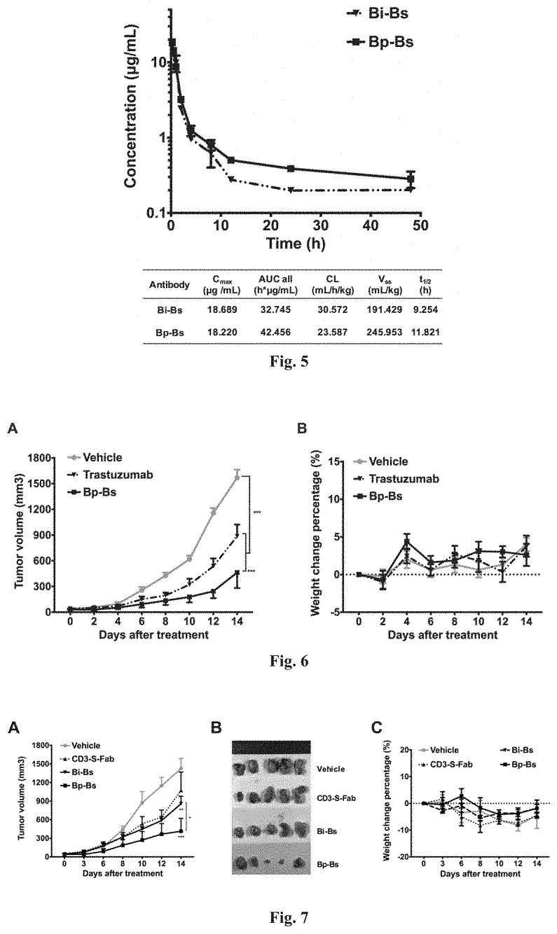

Results. The pharmacokinetic parameters were shown in FIG. 5. Bp-Bs showed a slightly higher residual concentration 10 hours after injection, the elimination half-lives of Bi-Bs and Bp-Bs in SCID mice were similar, suggesting that the VHH modification at the C-terminus of the Fab structure does not significantly affect the metabolism of Bp-Bs and Bi-Bs in vivo.

### Example 6. In Vivo Study on Anti-Tumor Activity of Bp-Bs

Methods: For in vivo xenograft studies, LS174T human colon cancer cells were harvested from cell culture, washed twice with PBS, and then resuspended in PBS. A total volume of 200 μl per mouse, containing 1×10⁶ LS174T cells, was injected subcutaneously into the right hind limb of NOD/SCID mice. When the tumor size reached 50 to 100 mm³, the mice were randomly divided into groups, with 5 or 6 animals in each group. 5×10⁶ freshly isolated human PBMCs (prepared according to the method in Example 3) were intraperitoneally administered. The animals were then treated with different doses of the antibody or control vehicle. The mice were weighed, and the tumor volume was measured in two vertical dimensions, and calculated using the following formula: (length×width²)/2. The mice were sacrificed when the tumor volume reached 1500 mm³. All results were expressed as the arithmetic mean of each group.

Results

Mice were divided into groups (n=6) and then treated with Bp-Bs at a dose of 1 mg/kg. The trastuzumab treatment group at a dose of 2 mg/kg was used as a positive control, and the PBS vehicle group was used as a negative control. Drugs were intraperitoneally injected every two days. After five treatments, on the 14th day after administration, the mean tumor volume was 1568 mm³ in the vehicle group, 886 mm³ in the 2 mg/kg trastuzumab treatment group, and 551 mm³ in the 1 mg/kg Bp-Bs treatment group. That is, 1 mg/kg of Bp-Bs can inhibit 65% of tumor growth. Compared with the clinically used Her2 monoclonal antibody trastuzumab, Bp-Bs has a significantly stronger anti-tumor effect (FIG. 6A). At the same time, there was no significant difference in the weight change of mice in each group, suggesting that Bp-Bs has no obvious side effects in this model (FIG. 6B).

In vitro experiments showed that Bp-Bs recruited T cells to specifically kill Her2 positive tumor cells, especially Her2 weakly expressing tumor cells, stronger than Bi-Bs. This inspired us to explore whether Bp-Bs can inhibit tumor growth more effectively in mouse tumor-bearing models. As previously reported, a human colon cancer subcutaneous tumor-bearing model on NOD/SCID mice was constructed and human immune system was established in the mice. The bispecific antibody Bi-Bs that binds bivalently to a single Her2 site and a bispecific antibody CD3-S-Fab that binds monovalently to a single Her2 site (see, Lin, L., L. Li, C. Zhou. J. Li, J. Liu, R. Shu, et al. (2018). “A Her2 bispecific antibody can be efficiently expressed in Escherichia coli with potent cytotoxicity.” Oncol Lett 16(1): 1259-1266) was added to the experiment for comparison. The model mice were treated with Bp-Bs, Bi-Bs or CD3-S-Fab at a dose of 1.5 mg/kg. The PBS vehicle group was set as a negative control. The mice were injected intraperitoneally once every three days for five times.

As shown in FIG. 7A, on the 14th day after administration, the mean tumor volume was 1424 mm³ in the vehicle group, 1073 mm³ in the CD3-S-Fab group, 857 mm³ in the Bi-Bs group, and 413 mm³ in the Bp-Bs group, suggesting Bp-Bs can inhibit tumor growth more effectively than Bi-Bs or CD3-S-Fab. The statistical results showed that on the 14th day after administration, the tumor volume sizes of the Bp-Bs group and the Bi-Bs group was significantly different from that of the vehicle group, and the paired T test results showed that the tumor sizes of the Bp-Bs group and the Bi-Bs group were significantly different (P<0.05). At the end point of the experiment, the mice subcutaneous tumors were dissected (FIG. 7B). In the Bp-Bs treatment group, there were 2 mice with extremely small tumor tissues, suggesting that 40% of the tumors may be completely suppressed by Bp-Bs administration. The administration of Bp-Bs, Bi-Bs or CD3-S-Fab did not cause significant changes in the body weight of the mice (FIG. 7C). These results indicated that in this animal tumor-bearing model, Bp-Bs exhibited a stronger tumor suppressive effect than Bi-Bs at the same dose. Bp-Bs can more effectively accumulate in Her2-positive tumor tissues, and then recruit more T cells to kill tumor cells.

All patents and other references cited in the specification are representations of the level of those of ordinary skill in the art to which the present invention belongs, and they are incorporated herein in their entirety by reference, including any tables and drawings therein, just like each document is individually incorporated herein by reference in their entirety. Those skilled in the art will easily realize that the present invention can be easily modified to obtain the objectives and advantages described herein and those implicit in this text. The methods, variants, and compositions described herein as representative of the currently preferred embodiments are exemplary and are not intended to limit the scope of the present invention. For those skilled in the art, they can be changed or used for other purposes, but these are all included in the scope of the present invention as defined by the appended claims.

1. A bispecific antibody with dual Her2 binding sites, comprising (a) an anti-CD3 antigen-binding fragment Fab, having a light chain variable region VL, a light chain constant region CL, a heavy chain variable region VH and a heavy chain constant region CH1;
(b) an anti-Her2 single domain antigen-binding fragment VHH1, linked to the C-terminus of the CL of the Fab and can bind to a first Her2 epitope; and
(c) an anti-Her2 single domain antigen-binding fragment VHH2, linked to the C-terminus of CH1 of the Fab and can bind to a second Her2 epitope;
wherein the first Her2 epitope and the second Her2 epitope are non-overlapping epitopes of Her2.

2. The bispecific antibody with dual Her2 binding sites of claim 1, wherein the VHH1 and/or VHH2 are linked to the Fab via a linker (GGGGS)₃.

3. The bispecific antibody with dual Her2 binding sites of claim 1, wherein the VHH1 and VHH2 have an amino acid sequence independently selected from a group consisting of a sequence comprising SEQ ID NO. 1, a sequence comprising SEQ ID NO. 2, and a sequence having more than 70% identity with any of the sequences.

4. The bispecific antibody with dual Her2 binding sites of claim 1, wherein the VHH1 and VHH2 have an amino acid sequence independently selected from a group consisting of SEQ ID NO. 1, SEQ ID NO. 2, and a sequence having more than 70% identity with any of SEQ ID NO. 1 or 2.

5. The bispecific antibody with dual Her2 binding sites of claim 1, wherein the anti-CD3 antigen-binding fragment Fab is an antigen-binding fragment derived from the CD3 monoclonal antibody UCHT1.

6. The bispecific antibody with dual Her2 binding sites of claim 1, wherein the bispecific antibody has a molecular weight of 60-100 kDa.

7. The bispecific antibody with dual Her2 binding sites of claim 1, wherein the bispecific antibody has a molecular weight of 79 kDa.

8. A bispecific antibody with dual Her2 binding sites, comprising
a first polypeptide chain, comprising a light chain constant region CL of an anti-CD3 Fab, a light chain variable region VL of the anti-CD3 Fab, and an anti-Her2 single domain antigen-binding fragment VHH1, wherein the VL, CL, VHH1 are linked sequentially from the N terminal to the C terminal, and
a second polypeptide chain, comprising a heavy chain constant region CH1 of the anti-CD3 Fab, a heavy chain variable region VH of the anti-CD3 Fab, and an anti-Her2 single domain antigen-binding fragment VHH2, wherein the VH, CH1, and VHH2 are linked sequentially from the N terminal to the C terminal;
wherein the first polypeptide chain and the second polypeptide chain are linked by a disulfide bond.

9. The bispecific antibody with dual Her2 binding sites of claim 8, wherein the first polypeptide chain has an amino acid sequence comprising a sequence shown in SEQ ID NO. 3 or a sequence having more than 90% identity with the sequence shown in SEQ ID NO. 3.

10. The bispecific antibody with dual Her2 binding sites of claim 8, wherein the second polypeptide chain has an amino acid sequence comprising a sequence shown in SEQ ID NO. 5 or a sequence having more than 90% identity with the sequence shown in SEQ ID NO. 5.

11. A pharmaceutical composition for tumor immunotherapy, comprising a therapeutically effective amount of a bispecific antibody with dual Her2 binding sites according to claim 1 and a pharmaceutically acceptable carrier.

12.-20. (canceled)

21. A method for treating a tumor in a subject, comprising administering to the subject a therapeutically effective amount of the bispecific antibody with dual Her2 binding sites of claim 1, or the pharmaceutical composition of claim 11.

22. The method of claim 21, wherein the tumor is a Her2 positive tumor with an IHC score of 1+, 2+ or 3+ as determined by immunohistochemistry.

23. The method of claim 21, wherein the tumor is selected from a group consisting of esophageal cancer, stomach cancer, colon cancer, rectal cancer, pancreatic cancer, lung cancer, breast cancer, cervical cancer, corpus cancer, ovarian cancer, bladder cancer, head and neck cancer, endometrial cancer, osteosarcoma, prostate cancer, and neuroblastoma.

24. The method of claim 21, wherein the tumor is a trastuzumab resistant or non-responsive tumor.
